(19) United States
(12) Patent Application Publication
(10) Pub. No.: US 2010/0203010 A1
(43) Pub. Date: Aug. 12, 2010

(54) MODULATION OF TUMOR MICROENVIRONMENT

(75) Inventors: Kandasamy Hariharan, San Diego, CA (US); Shabnam Tangri, San Diego, CA (US); Arturo Molina, San Diego, CA (US)

Correspondence Address: PILLSBURY WINTHROP SHAW PITTMAN LLP, P.O. BOX 10500, MCLEAN, VA, 22102, US

(73) Assignee: BIOGEN IDEC INC., Cambridge, MA (US)

(21) Appl. No.: 12/593,231

(22) PCT Filed: Mar. 28, 2008
(86) PCT No.: PCT/US08/58744

§ 371 Date: Apr. 16, 2010

Related U.S. Application Data
(60) Provisional application No. 60/908,645, filed on Mar. 28, 2007.

(51) Int. Cl. A61K 38/20; A61K 39/395; A61P 35/00; A61K 38/21

(52) U.S. Cl. 424/85.2; 424/173.1; 424/133.1; 424/85.5

(57) ABSTRACT

Compositions comprising CD80-targeted therapeutics and methods of using these compositions are provided for the treatment of a disease or disorder in which CD80-expressing cells or regulatory T cell function contribute to or exacerbate the associated pathology.

### RELATED APPLICATIONS

Priority is claimed from U.S. Provisional Application No. 60/908,645, filed Mar. 28, 2007, which is incorporated herein by reference in its entirety.

### FIELD OF THE INVENTION

The present invention generally relates to cancer therapies based upon modulation of immune cells in the tumor microenvironment. More particularly, the present invention relates to therapeutics that target or alter the function of non-malignant cells that promote tumor growth and survival.

### BACKGROUND OF THE INVENTION

Cells of the innate and adaptive immune systems are poised to rapidly respond to tissue injury and/or pathogenic infection. The balance between quiescence and activation of immune and inflammatory responses is a highly regulated equilibrium that controls anti-pathogen and anti-tumor immune responses. Cell surface and secreted proteins regulate this equilibrium through both positive and negative feedback mechanisms designed to ensure an appropriately rapid response while minimizing deleterious consequences.

In the context of cancer, the unique, non-physiologic tissue microenvironment that constitutes a tumor is composed of both malignant and non-malignant cells. While transforming or oncogenic alterations in the malignant cells clearly underlie unregulated growth and tumor progression, non-malignant cells and the tumor microenvironment, which results from the juxtaposition of malignant and non-malignant cells, play a critical role in tumor initiation. Non-malignant cells and the tumor microenvironment are also important for tumor progression, maintaining conditions that support further genetic instability and elevated mutation frequencies. See e.g., Reynolds et al., Cancer Res., 1996, 56(24):5754-5757. In particular, non-malignant cells that function normally to support inflammatory and immune response are, in certain circumstances, within a tumor microenvironment capable of contributing to tumor progression, for example, by producing mediators that directly or indirectly support growth and viability of malignant cells within the tumor, or by producing mediators that directly or indirectly inhibit the growth and viability of malignant cells, or by inhibiting responses that would otherwise impede tumor progression. The tumor microenvironment also influences accessibility of a tumor to therapeutic intervention by altering drug metabolism or pharmacokinetics at the tumor site and/or contributing to the development of drug resistance.

Despite a growing understanding of the importance of the tumor microenvironment, therapeutic strategies targeting this biology remain limited. As one example, patients with multiple myeloma have been treated with a proteasome inhibitor, which targets bone marrow stroma, to thereby reduce bone metastasis. See Rajkumar et al., J. Clin. Oncol., 2005, 20; 23(3):630-399. Additional examples include drugs and candidate therapeutic agents that modulate angiogenesis, hypoxia, and chemokines. See e.g., Nagasawa et al., Biol. Pharm. Bull., 2006, 29(12):2335-2342; Giles et al., Curr. Cancer Drug Targets, 2006, 6(8):659-670.

In view of the continuing need for anti-cancer therapies, the present invention provides methods of modulating the function of non-malignant cells present in the tumor microenvironment to thereby disrupt conditions that support tumor growth and survival. In particular, the disclosed methods include administering a CD80-targeted therapeutic agent to elicit immunomodulatory effects, including depletion of immunoregulatory or inflammatory cells such as antigen presenting cells (e.g., macrophages, dendritic cells, B cells) or myeloid-derived suppressor cells present within the tumor microenvironment, and inhibition of T cell subsets that function to support tumor progressions (e.g., regulatory T cells and Th2 helper cells). The disclosed methods are also relevant to the treatment of additional disorders in which regulatory T cell suppression contributes to deterioration of a pathological condition.

### SUMMARY OF THE INVENTION

The present invention provides methods of modulating non-malignant cells in a tumor microenvironment to thereby inhibit tumor progression. For example, the present invention provides a method of treating a subject having a malignancy, which comprises a tumor microenvironment of malignant and non-malignant cells, wherein regulatory T cell function contributes to or exacerbates the malignancy, comprising administering to the subject a therapeutically effective amount of a CD80-targeted therapeutic. In another aspect of the invention, methods are provided for treating a subject having a malignancy, which comprises a tumor microenvironment of malignant and non-malignant cells, wherein non-malignant CD80-expressing cells contribute to or exacerbate the malignancy, comprising administering to the subject a therapeutically effective amount of a CD80-targeted therapeutic. CD80-targeted therapeutics as described herein may also be used to suppress production of one or more inflammatory cytokines in a tumor microenvironment of a subject and/or to deplete non-malignant CD80-expressing cells in a tumor microenvironment of a subject.

### BRIEF DESCRIPTION OF THE DRAWINGS

FIG. 1A depicts the percentage of CD4+CD25− T cells proliferating when co-cultered with irradiated SB lymphoma cells in the presence of no treatment (control, diamonds) CTLA-4Ig (triangles), anti-CD86 antibody (gray circles), anti-CD80 antibody (galiximab, open circles), or a combination of anti-CD86 and anti-CD80 antibodies (black circles).

FIG. 1B depicts the percentage of CD4+CD25− T cells proliferating when co-cultered with LPS stimulated mature dendritic cells in the presence of no treatment (control, diamonds) CTLA-4Ig (triangles), anti-CD86 antibody (gray circles), anti-CD80 antibody (galiximab, open circles), or a combination of anti-CD86 and anti-CD80 antibodies (black circles).

FIG. 2A depicts the production of IFN-γ by CD4+CD25− T cells when co-cultered with LPS stimulated mature dendritic cells in the presence of no treatment (control, diamonds), CTLA-4Ig (triangles), anti-CD86 antibody (gray circles), anti-CD80 antibody (galiximab, open circles), or a combination of anti-CD86 and anti-CD80 antibodies (black circles).

FIG. 2B depicts the production of interleukin-2 by CD4+CD25− T cells when co-cultered with LPS stimulated mature dendritic cells in the presence of no treatment (control, diamonds), CTLA-4Ig (triangles), anti-CD86 antibody (gray circles), anti-CD80 antibody (galiximab, open circles), or a combination of anti-CD86 and anti-CD80 antibodies (black circles).

FIG. 3A depicts the percentage of regulatory T cell induction of CD4+CD25+ T cells of Donor 202 when co-cultered with irradiated SB lymphoma cells at a SB:T ratio of 1:120 in the presence of no treatment (control, diamonds), human IgG1 (isotype control, squares), CTLA-4Ig (triangles), anti-CD86 antibody (X), anti-CD80 antibody (galiximab, open circles), and a combination of anti-CD86 and anti-CD80 antibodies (black circle). Also shown is the percentage of regulatory T cell induction of CD4+CD25+ T cells of Donor 202 when co-cultered with irradiated SB lymphoma cells at a SB:T ratio of 1:10 (plus signs) and no co-culture, i.e., culture of CD4+CD25+ T cells alone (rectangles).

FIG. 3B depicts the percentage of regulatory T cell induction of CD4+CD25+ T cells of Donor 133 when co-cultered with irradiated SB lymphoma cells at a SB:T ratio of 1:160 in the presence of no treatment (control, diamonds), human IgG1 (isotype control, squares), CTLA-4Ig (triangles), anti-CD86 antibody (X), anti-CD80 antibody (galiximab, open circles), and a combination of anti-CD86 and anti-CD80 antibodies (black circle). Also shown is the percentage of regulatory T cell induction of CD4+CD25+ T cells of Donor 202 when co-cultered with irradiated SB lymphoma cells at a SB:T ratio of 1:10 (plus signs) and no co-culture, i.e., culture of CD4+CD25+ T cells alone (rectangles).

FIGS. 4A-4F depict interleukin-8 (FIGS. 4A, 4C, and 4E) and interleukin-6 (FIGS. 4B, 4D, and 4F) production by CD14+ monocytes (open bars), CD14+ monocytes co-cultured with Raji lymphoma cells (gray bars), CD4+ T cells co-cultured with CD14+ monocytes (hatched bars), and CD4+ T cells co-cultured with CD14+ monocytes and Raji lymphoma cells (black bars), in the presence of purified human serum myeloma IgG1 (isotype control), anti-CD80 antibody (galiximab), or CTLA-4Ig. Cell populations were isolated from Donor A (FIGS. 4A-4B), Donor B (FIGS. 4C-4D), and Donor C (FIGS. 4E-4F).

### DETAILED DESCRIPTION

The present invention provides methods for modulation of non-malignant cells within the tumor microenvironment, which normally function to regulate inflammation as well as innate and adaptive immunity, and which directly or indirectly support tumor progression. The disclosed methods are also useful for promoting cancer immunity, i.e., anti-cancer activity of cancer-reactive T cells. The disclosed methods employ a CD80-targeted therapeutic which is effective in blocking CD80-mediated regulation of T cell function or homeostasis and/or by depletion of CD80-expressing non-malignant cells that support tumor growth, migration, and/or angiogenesis. In one aspect of the invention, methods are provided for treating a subject having a disease or disorder in which regulatory T cell function contributes to the disease pathology by administering to the subject a therapeutically effective amount of a CD80-targeted therapeutic. Representative indications include cancer, including both hematological as well as non-hematological malignancies. In other aspects of the invention, methods are provided for using a CD80-targeted therapeutic for enhancing cancer immunity or reducing immune or inflammatory support of tumor progression in a subject by modulating immune cells present in the tumor microenvironment, for example, by decreasing the number of immunoregulatory or inflammatory cells such as antigen presenting cells (e.g., macrophages, dendritic cells, B cells) or myeloid-derived suppressor cells (e.g., myeloid-derived monocytes and tie-2-expressing monocytes) present within the tumor microenvironment, inhibiting T cell subsets that function to support tumor progression (e.g., regulatory T cell and Th2 helper T cells), and/or suppressing production of one or more inflammatory cytokines in a tumor microenvironment.

### I. The Tumor Microenvironment

The present invention is based upon the modulation of cells that either directly or indirectly contribute to disease pathogenesis or progression/persistence, wherein regulatory T cell function contributes to disease. In one aspect of the invention, non-malignant cells of a tumor microenvironment are modulated such that the direct contribution of those cells to tumor progression is inhibited, and the function of those cells in supporting regulatory T cell function is also inhibited.

Cells of a tumor microenvironment comprise malignant cells in association with non-malignant cells that support their growth and survival. The non-malignant cells, also called stromal cells, occupy or accumulate in the same cellular space as malignant cells, or the cellular space adjacent or proximal to malignant cells, which modulate tumor cell growth or survival. Non-malignant cells of the tumor microenvironment include fibroblasts, myofibroblasts, glial cells, epithelial cells, adipocytes, vascular cells (including blood and lymphatic vascular endothelial cells and pericytes), resident and/or recruited inflammatory and immune (e.g., macrophages, dendritic cells, myeloid suppressor cells, granulocytes, lymphocytes, etc.), resident and/or recruited stem cells that are capable of giving rise to or differentiating into any of the above-noted non-malignant cells, and any functionally distinct subtypes of the above-noted cells as known in the art. These cells actively participate in tumor cell growth and metastasis.

In performing the methods disclosed herein, a tumor microenvironment is identified using one or more of the following criteria: (a) a region comprising non-malignant cells which share the same physiological environment, or which are directly adjacent to malignant cells; (b) the extended tumor region; (c) an area of inflammation surrounding or proximal to a tumor; (d) an area in which the number or rate of proliferation of regulatory T cells is elevated; and (e) an area in which macrophages, dendritic cells, or myeloid-derived suppressor cells are elevated. Within the context of non-solid tumor types, the tumor microenvironment may also be determined by the local cell-cell interactions between malignant cells and between malignant cells and any adjacent or nearby non-malignant cells. Such interactions may include, for example, cell adhesion events and/or paracrine effects of soluble mediators produced by one cell (malignant or non-malignant) on another cell (malignant or non-malignant) in the tumor microenvironment.

When assessing a level of any one of the above-noted criteria, e.g., a level of inflammation or a number of regulatory T cells, the level is assessed relative to a reasonable control level. For example, a relevant control may comprise a sample taken from a tumor-bearing subject and from a same tissue and analogous region on the contralateral side of the subject. As another control, a sample may be taken from a same tissue and analogous region from a similarly situated (age, gender, overall health, etc.) subject who lacks a tumor. In the case of assessment of treatment-dependent response, post-treatment effects may also be ascertained through parallel analysis of a pre-treatment control sample.

When quantifying a level of any of the above-described criteria for defining a tumor microenvironment, a difference when assessed relative to a control level is identified as a difference of at least about two-fold greater or less than a control level, or at least about five-fold greater or less than a control level, or at least about ten-fold greater or less than a control level, at least about twenty-fold greater or less than a control level, at least about fifty-fold greater or less than a control level, or at least about one hundred-fold greater or less than a control level. A difference in the above-noted criteria when assessed relative to a control level may also be observed as a difference of at least 20% compared to a control level, such as at least 30%, or at least 40%, or at least 50%, or at least 60%, or at least 70%, or at least 80%, or at least 90%, or at least 100%, or more. For example, an elevated number of regulatory T cells may be assessed relative to a known physiological level of regulatory T cells in a patient lacking a tumor. Based upon the foregoing, one of ordinary skill in the art (e.g., a clinician in oncology) would be able to readily identify a region constituting a tumor microenvironment in a patient using the above-noted criteria and selection of appropriate controls. A subject may also have one or more tumor microenvironments, each associated with a distinguishable tumor. The characteristics of a tumor microenvironment may vary in relation to the tissue site of the tumor, the grade of a tumor, the stage of a tumor, the morphological or molecular phenotype of a tumor, etc. A tumor microenvironment may also change as a tumor progresses.

Among the non-malignant cells of a tumor microenvironment are regulatory T cells, which are observed in higher frequencies in a number of tumors, including Hodgkin's lymphoma, non-Hodgkin's lymphoma (Shi et al., Ai Zheng., 2004, 23(5):597-601 (abstract only)), malignant melanoma (Viguier et al., J. Immunol., 2004, 173(2):1444-53; Javia et al., J. Immunother., 2003, 26(1):85-93), and cancers of the ovary (Woo et al., Cancer Res., 2001, 61(12):4766-72), gastrointestinal tract (Ichihara et al., Clin Cancer Res., 2003, 9(12):4404-4408; Sasada et al., Cancer, 2003, 98(5):1089-1099), breast (Liyanage et al., J Immunol., 2002, 169(5):2756-2761), lung (Woo et al., Cancer Res., 2001, 61(12):4766-72), and pancreas (Liyanage et al., J Immunol., 2002, 169(5):2756-2761). The regulatory T cells are recruited to the tumor site in response to chemokines secreted by the tumor cells. See e.g., Curiel et al., Nat. Med., 2004, 10:942-949. An increase in the number of regulatory T cells may also correlate with poor prognosis (Curiel et al., Nat. Med., 2004, 10:942-949; Sasada et al., Cancer, 2003, 98:1089-1099). Conversely, regulatory T cells are observed to decrease following chemotherapy (Beyer et al., Blood, 2005, 106:2018-2025).

The presence of tumor-associated macrophages has also been shown to correlate with poor prognosis and survival in tumors such as those of the breast, ovary, prostrate, cervix, and lung (Pollard, Nat. Rev. Cancer, 2004, 4:71-77). A similar correlation was observed for patients with follicular lymphoma (Farinha et al., Blood, 2005, 15:2169-2174). Similarly, myeloid-derived suppressor cells have been suggested to play a role in regulating immune cell-mediated responses.

The disclosed methods target non-malignant immune cells of the tumor microenvironment as an opportunity for therapeutic intervention. In one aspect of the invention, methods are provided for inhibiting regulatory T cell and Th2 helper T cell function. Such inhibition constitutes a reduction in the number of T cells and/or disruption of the functional capability of T cells to suppress anti-tumor responses. For example, relevant functions of regulatory T cells include production of pro-inflammatory cytokines, such as interleukin-2, interleukin-4, interleukin-10, interleukin-12, transforming growth factor beta, and interferon-γ. See e.g., Mocellin et al., J. Immunother., 2001, 24(5):3920407. The resultant inflammatory tumor microenvironment attracts macrophages that in turn provide malignant cells with trophic or survival signals, promote angiogenesis, alter the phenotype and function of antigen presenting cells and dendritic cells, amplify regulatory T cells, which collectively result in an immunosuppressed cellular environment. Relevant functions of Th2 helper cells similarly include production of signals that promote survival of associated malignant cells, for example, CD40/CD40L signaling, interleukin-1, or interleukin-6. Th2 helper cells also contribute to the inflammatory tumor microenvironment by secretion of chemokines that recruit antigen presenting cells, including macrophages.

Regulatory T cells may be identified as enriched within the CD4⁺CD25^{hi} population and may be further characterized by expression of cytotoxic T lymphocyte-associated antigen 4 (CTLA4), glucocorticoid-induced tumor necrosis factor receptor family-related gene (GITR), CC chemokine receptor 4 (CCR4), forkhead box p3 (FOXP3), CC chemokine receptor 6 (CCR6), and/or CD30. In addition, regulatory T cells may be CD62L^{hi}, CD45RB^{lo}, CD45RO^{hi}, and/or CD45RA⁻. See McHugh et al., J. Allergy Clin. Immunol., 2002, 110:693-701; Hon et al., Science, 2003, 299:1057-1061; Iellem et al., J. Exp. Med., 2001, 194:847-853; U.S. Patent Application Publication No. 2006/0063256.

Helper T cells include CD4⁺CD25⁻ Th1 cells and Th2 cells. Th1 cells promote cellular immunity, i.e., the induction of antigen-specific CD8⁺ cytotoxic T cells. Th2 helper T cells can suppress Th1 cell-mediated cellular immunity, for example, by secreting cytokines that inhibit interferon-γ production by Th1 cells (Fiorentino et al., J. Exp., Med., 1989, 170: 2081-2095).

CD80-CD28 signaling is a major coregulatory pathway for regulatory T cell and Th2 helper cell differentiation and maintenance, and T cells dramatically decrease in the absence of CD80 costimulation. See Zheng et al., J. Immunol., 2004, 172:2778-2784; Liang et al., J. Exp. Med., 2005, 201: 127-137; and Tang et al., J. Immunol., 2003, 171:3348-3352. As described herein, useful CD80-targeted therapeutics include molecules that block CD80/CD28 signaling to thereby attenuate the immunosuppressive effects of regulatory T cells and Th2 helper T cells, which then permits tumor-reactive T cells to mediate anti-tumor activity. Selective blockade of CD80/CD28 signaling, i.e., in the absence of blockade of CD80/CTLA4 signaling, may offer improved therapeutic efficacy given that the negative modulation of immune responses by CD80/CTLA4 signaling is not disrupted.

The activation of regulatory T cells and Th2 helper T cells creates an inflammatory tumor microenvironment that attracts macrophages. The macrophages promote survival of malignant cells by secreting cytokines such as IL-1 and IL-6. Macrophages also secrete chemokines for the recruitment of additional immune effector cells to the tumor microenvironment, for example, IL-8, which attracts neutrophils, and MIP-1α, which attracts leukocytes. Macrophages further secrete angiogenic factors, which contribute to vascularization of the tumor.

CD80-targeted therapeutics may also bind directly CD80 expressed on antigen presenting cells of the tumor microenvironment. Accordingly, they may be depleted by induction of antibody dependent cellular cytotoxicity (ADCC), complement dependent cytotoxicity, and/or apoptosis. Relevant methods for assessing these activities are well known in the art.

Representative methods for assessing changes in non-malignant immune cells of the tumor microenvironment are described herein below with respect to determination of a therapeutically effective dose and in the Examples.

### II. CD80-Targeted Therapeutics

The CD80-targeted therapeutics of the invention encompass molecules that block activation of regulatory T cells and Th2 helper cells and/or deplete CD80-expressing cells in a tumor microenvironment. Depletion may occur by any mechanism for inducing cell destruction, including induction of apoptosis, inhibition or reduction of cell growth, and/or antibody-dependent cell-mediated cytotoxicity (ADCC). Thus, CD80-targeted therapeutics include CD80 antagonists as well as CD80-binding molecules conjugated to a cytotoxic agent. CD80-targeted therapeutics useful in the invention include any synthetic, recombinant, or natural product or composition having the above-noted properties. Representative agents include but are not limited to peptides, proteins, nucleic acids (e.g., aptamers), small molecules (e.g., chemical compounds), antibodies, and nucleic acid-protein fusions.

For CD80-binding conjugates, cytotoxic agents may be associated or bound to CD80-binding molecules either directly or indirectly, for example, using linker molecules or biological or synthetic matrices. Cytotoxic agents can be covalently bound to CD80-binding molecules by techniques well known in the art such as the use of the heterobifunctional crosslinking reagents. The linker molecule may be a cleavable linker facilitating release of the cytotoxic drug in cells. For example, an acid labile linker, peptidase sensitive linker, dimethyl linker or disulfide containing linker may be used.

Useful cytotoxic agents are known in the art, including any of the agents described herein below as additionally useful in combination with a CD80-targeted therapeutic, such as radioactive isotopes, chemotherapeutic agents, and toxins such as small molecule toxins or enzymatically active toxins of bacterial, fungal, plant or animal origin, or fragments thereof. Additional representative cytotoxic agents include cytostatic agents, alkylating agents, antimetabolites, anti-proliferative agents, tubulin binding agents, hormones and hormone antagonists, and the like. Additional representative cytotoxins include members or derivatives of the enediyne family of anti-tumor antibiotics, including calicheamicin, esperamicins or dynemicins.

II.A. Anti-CD80 Antibodies

CD80-targeted therapeutics of the present invention include anti-CD80 antibodies or CD80-binding fragments thereof. Naturally occurring antibodies comprise two identical light polypeptide chains, each having a molecular weight of approximately 23,000 Daltons, and two identical heavy chains, each having a molecular weight of 53,000-70,000 Daltons. The four chains associate to form three globular domains joined by a flexible hinge region. The variable domains of both the light (V_{L}) and heavy (V_{H}) chains determine antigen recognition and specificity. The constant domains of the light chain (C_{L}) and the heavy chain (C_{H}1, C_{H}2 or C_{H}3) confer biological properties such as Fc receptor binding, complement binding and the like. Anti-CD80 antibodies useful in the present invention, may include polyclonal antibodies, monoclonal antibodies (mAbs), humanized or chimeric antibodies, PRIMATIZED® antibodies, which contain human constant regions and primate (cynomolgus macaque) variable regions, human monoclonal antibodies, single chain antibodies, Fab fragments, F(ab′)₂ fragments, fragments produced by a Fab expression library, anti-idiotypic (anti-Id) antibodies, and epitope-binding fragments of any of the above. Such antibodies may have a structure of a naturally occurring antibody, multivalent forms thereof, or fragments thereof.

Monoclonal antibodies, which are homogeneous populations of antibodies to a particular antigen, may be obtained by any technique that provides for the production of antibody molecules by continuous cell lines in culture. These include, but are not limited to the hybridoma technique of Kohler and Milstein, Nature, 1975, 256:495-497; and U.S. Pat. No. 4,376,110), the human B cell hybridoma technique (Kosbor et al., Immunology Today, 1983, 4:72; Cote et al., Proc. Natl. Acad. Sci. USA, 1983, 80:2026-2030), and the EBV-hybridoma technique (Cole et al., in Monoclonal Antibodies And Cancer Therapy, 1995, Alan R. Liss, Inc., New York, pp. 77-96). Such antibodies may be of any immunoglobulin class including IgG, IgM, IgE, IgA, IgD and any subclass thereof. Hybridomas producing monoclonal antibodies may be cultivated in vitro or in vivo.

A chimeric antibody is a molecule in which different portions are derived from different animal species, such as those having a variable region derived from a murine mAb and a human immunoglobulin constant region. Chimeric antibodies may be produced by splicing the genes from a mouse antibody molecule of appropriate antigen specificity together with genes from a human antibody molecule of appropriate biological activity (Morrison et al., Proc. Natl. Acad. Sci. USA, 1984, 81:6851-6855; Takeda et al., Nature, 1985, 314:452-454).

A humanized antibody is a type of chimeric antibody, wherein variable region residues responsible for antigen binding (i.e., residues of a complementarity determining region and any other residues that participate in antigen binding) are derived from a non-human species, while the remaining variable region residues (i.e., residues of the framework regions) and constant regions are derived, at least in part, from human antibody sequences. Residues of the variable regions and constant regions of a humanized antibody may also be derived from non-human sources. Variable regions of a humanized antibody are also described as humanized (i.e., a humanized light or heavy chain variable region). The non-human species is typically that used for immunization with antigen, such as mouse, rat, rabbit, non-human primate, or other non-human mammalian species. Humanized antibodies may be prepared using any one of a variety of methods including veneering, grafting of complementarity determining regions (CDRs), grafting of abbreviated CDRs, grafting of specificity determining regions (SDRs), and Frankenstein assembly, as described below. These general approaches may be combined with standard mutagenesis and synthesis techniques to produce a CD80 antibody of any desired sequence.

The antibodies of the present invention may also be human monoclonal antibodies, for example those produced by immortalized human cells, by SCID-hu mice or other non-human animals capable of producing human antibodies. Human antibodies may also be isolated from antibody phage libraries, for example, as described by Marks et al., J. Mol. Biol., 1991, 222:581-597. Chain shuffling and recombination techniques may be used to produce phage libraries having increased antibody diversity, e.g., libraries including antibodies with increased binding affinity. See Marks et al., Biotechnology, 1992, 10:779-783 and Waterhouse et al., Nuc. Acids Res., 1993, 21:2265-2266.

The antibodies of the present invention can also comprise single chain antibodies, which are typically formed by linking the heavy and light chain fragments of the Fv region via an amino acid bridge, resulting in a single chain polypeptide. See e.g., U.S. Pat. No. 4,946,778; Bird, Science, 1988, 242:423-426; Huston et al., Proc. Natl. Acad. Sci. USA, 1988, 85:5879-83; and Ward et al., Nature, 1989, 334:544-546).

Antibodies useful in the invention also include non-fucosylated antibodies. Such antibodies include an Fc region and complex N-glycoside-linked sugar chains bound to the Fc region, wherein among the total complex N-glycoside-linked sugar chains bound to the Fc region, the ratio of a sugar chain in which fucose is not bound to N-acetylglucosamine in the reducing end in the sugar chain is at least 20%. The non-fucosylated antibodies have enhanced Fc receptor binding and enhanced effector functions as compared to control fucosylated antibodies. See U.S. Provisional Application No. 60/908,643, filed Mar. 28, 2007, which is incorporated herein by reference in its entirety. Representative methods for production of non-fucosylated antibodies are set forth in Example 1. See also U.S. Patent Publication No. 2004/0093621, U.S. Pat. No. 6,946,292, PCT International Publication No. WO 2006/089232, and European Published Application No. 1176195, each of which is also incorporated herein by reference in its entirety.

Antibody fragments that recognize specific antigens or surface receptors as described herein may be generated by known techniques. For example, such fragments include F(ab′)₂ fragments or Fc regions that can be produced by pepsin digestion of the antibody molecule and Fab fragments that can be generated by reducing the disulfide bridges of the F(ab′)₂ fragments. Alternatively, Fab expression libraries may be constructed (Huse et al., Science, 1989, 246:1275-1281) to allow rapid and easy identification of monoclonal Fab fragments with the desired specificity.

Specific binding of an antibody or antibody fragment to the antigens described herein refers to preferential binding of an antibody to the antigen in a heterogeneous sample comprising multiple different antigens. Substantially lacking binding describes a level of binding of an antibody to a control protein or sample, i.e., a level of binding characterized as non-specific or background binding. The binding of an antibody to an antigen is specific if the binding affinity is at least about 10⁻⁷ M or higher, such as at least about 10⁻⁸ M or higher, including at least about 10⁻⁹ M or higher, at least about 10⁻¹¹ M or higher, or at least about 10⁻¹² M or higher.

A representative anti-CD80 antibody useful in the invention is galiximab, which is alternatively referred to as PRIMATIZED® 16C10, IDEC-114, or a PRIMATIZED® antibody having variable regions produced by the antibody produced by the hybridoma of American Type Culture Collection (ATCC) Accession No. HB-12119. HB-12119 hybridoma was deposited on May 29, 1996, with the ATCC, currently located at 10801 University Boulevard, Manassas, Va. 20110-2209, under the provision of the Budapest Treaty for the International Recognition of the Deposit of Microorganisms for the Purpose of Patent Procedure (“Budapest Treaty”). Galiximab is a PRIMATIZED® anti-CD 80 immunoglobulin (Ig) G1 lambda monoclonal antibody with human constant regions and primate (cynomologous macaque) variable regions. The amino acid and nucleic acid sequences of galiximab and other anti-CD80 antibodies useful in the invention are disclosed in U.S. Pat. No. 6,113,898, which is hereby incorporated by reference in its entirety.

Additional representative anti-CD80 antibodies or CD80-binding fragments thereof include antibodies and CD80-binding fragments thereof that compete with 16C10 or galiximab for binding to CD80 in a binding inhibition assay. Such binding inhibition assays are well-known to the skilled artisan. The present invention also includes anti-CD80 antibodies and CD80-binding fragments that may bind to the same CD80 epitope as 16C10 or galiximab. Anti-CD80 antibodies that compete for binding to CD80 with galiximab and other anti-CD80 antibodies, such as antibodies that may bind to the same epitope as galiximab, are disclosed in U.S. Pat. No. 7,153,508, which is hereby incorporated by reference in its entirety. Methods for determining the binding specificity of antibodies can be determined by immunoprecipitation or by an in vitro assay, such as a radioimmunoassay (RIA) or enzyme-linked immunoabsorbent assay (ELISA) as are well known to the skilled artisan.

The present invention further includes those anti-CD80 antibodies or CD80-binding fragments thereof, which comprise the variable regions of galiximab, or variable regions derived from galiximab variable regions, for example, by introduction of one or more amino acid additions, substitutions, or other mutations. Additional anti-CD80 antibodies and CD80 binding fragments useful in the invention include antibodies having residues comprising the antigen binding domain of galiximab, i.e., the complementarity determining regions of galiximab.

Antibodies useful in the invention may be recombinantly prepared using standard techniques. See e.g., Harlow & Lane, Antibodies: A Laboratory Manual, 1988, Cold Spring Harbor Laboratory Press, Cold Spring Harbor, N.Y. and U.S. Pat. Nos. 4,196,265; 4,946,778; 5,091,513; 5,132,405; 5,260,203; 5,658,570; 5,677,427; 5,892,019; 5,985,279; 6,054,561. Representative methods for producing antibodies comprising 16C10 variable regions are described in U.S. Pat. No. 6,113,898. See also U.S. Patent Application Publication Nos. 20030103971 and 20030180290.

Tetravalent antibodies (H₄L₄) comprising two intact tetrameric antibodies, including homodimers and heterodimers, may be prepared, for example, as described in PCT International Publication No. WO 02/096948. Antibody dimers may also be prepared via introduction of cysteine residue(s) in the antibody constant region, which promote interchain disulfide bond formation, using heterobifunctional cross-linkers (Wolff et al., Cancer Res., 1993, 53:2560-2565), or by recombinant production to include a dual constant region (Stevenson et al., Anticancer Drug Des., 1989, 3:219-230.

II.B. Small Molecules

The present invention also provides small molecules which may be used as CD80-targeted therapeutics. These molecules include heterocyclic compounds of formula (I) as described below and in U.S. patent application Ser. No. 11/539,153. Such compounds inhibit the interaction between CD80 and CD28 as required for regulatory T cell and Th2 helper cell activation. Examples of heterocyclic compounds that may be employed in the inventive methods include the compounds of formula (I) or a pharmaceutically acceptable salt thereof. These heterocyclic compounds of formula (I) are prepared essentially as described in U.S. Pat. No. 7,081,456, which is herein incorporated by reference in its entirety. Formula (I) is depicted as follows:

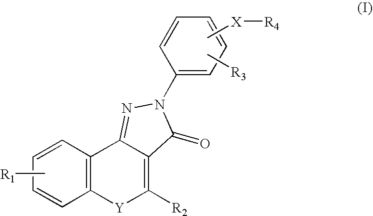

wherein

R₁ and R₃ independently represent H; F; Cl; Br; —NO₂; —CN; C₁ C₆ alkyl optionally substituted by F or Cl; or C₁ C₆ alkoxy optionally substituted by F;

R₂ represents H, or optionally substituted C₁ C₆ alkyl, C₃ C₇ cycloalkyl or optionally substituted phenyl;

Y represents —O—, —S—, N-oxide, or —N(R₅)— wherein R₅ represents H or C₁ C₆ alkyl;

X represents a bond or a divalent C₁ C₆ alkylene radical;

R₄ represents —C(═O)NR₆R₇, wherein R₆ represents a radical of formula -(Alk)_{b}-Q wherein b is 1 and Alk is an optionally substituted divalent straight chain or branched C₁ C₁₂ alkylene, C₂ C₁₂ alkenylene or C₂ C₁₂ alkynylene radical which may be interrupted by one or more non-adjacent —O—, —S— or —N(R₈)— radicals wherein R₈ represents H or C₁ C₄ alkyl, C₃ C₄ alkenyl, C₃ C₄ alkynyl, or C₃ C₆ cycloalkyl, and

Q represents H; —CF₃; —OH; —SH; —NR₈R₈ wherein each R₈ may be the same or different; an ester group; or an optionally substituted phenyl, C₃ C₇ cycloalkyl, C₅ C₇ cycloalkenyl or heterocyclic ring having from 5 to 8 ring atoms; and

R₇ represents H or C₁ C₆ alkyl; or when taken together with the atom or atoms to which they are attached R₆ and R₇ form an optionally substituted heterocyclic ring having from 5 to 8 ring atoms.

Representative molecules of formula (I) include fused pyrazolones having the following structures A and B:

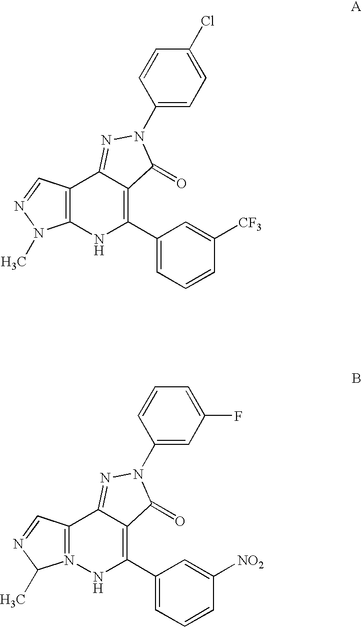

### III. Therapeutic Applications

As described herein, CD80-targeted therapeutics may be used for modulation of T cells associated with pathologic conditions deteriorated by regulatory T cell and/or Th2 helper cell function. CD80-targeted therapeutics may also be used for depletion of immunoregulatory and inflammatory cells in the tumor microenvironment (e.g., antigen presenting cells and myeloid-derived suppressor cells), which cells support tumor growth, migration, angiogenesis, etc. Thus, CD80-targeted therapeutics as described herein act to enhance adaptive immune responses, including inhibition of the suppressive functions of regulatory T cells. The CD80-targeted therapeutics may be used alone or in combination with additional therapeutic agents as described further below. Subjects may receive CD80-targeted therapeutics as a first line of therapy, or for treatment of relapsed or refractory conditions.

III.A. Indications

In one aspect of the invention, CD80-targeted therapeutics may be used for the treatment of hematologic or non-hematologic malignancies by targeting non-malignant cells of a tumor microenvironment. The CD80-targeted therapeutics are similarly useful for inhibiting growth of cells of a non-malignant proliferative disorder, such as hyperplasia, metaplasia, or most particularly, dysplasia (for review of such abnormal growth conditions, see DeVita, Jr. et al., Cancer: Principles and Practice, 6th edition, 2001, Lippincott Williams & Wilkins.

For example, CD80-targeted therapeutics as described herein may be used to treat B cell malignancies, e.g., leukemias and lymphomas, including indolent, aggressive, low-grade, intermediate-grade, or high-grade leukemia or lymphoma. Representative B cell malignancies include Hodgkin's lymphoma, B cell chronic lymphocytic leukemia (B-CLL), lymhoplasmacytoid lymphoma (LPL), mantle cell lymphoma (MCL), follicular lymphoma (FL), diffuse large cell lymphoma (DLCL), Burkitt's lymphoma (BL), AIDS-related lymphomas, monocytic B cell lymphoma, angioimmunoblastic lymphoadenopathy, small lymphocytic; follicular, diffuse large cell; diffuse small cleaved cell; large cell immunoblastic lymphoblastoma; small, non-cleaved: Burkitt's and non-Burkitt's: follicular, predominantly large cell; follicular, predominantly small cleaved cell; and follicular, mixed small cleaved and large cell lymphomas. Subjects having any of the above-identified B cell malignancies include relapsed subjects, or subjects who are refractory to prior therapy.

Additional cancers that may be treated using the disclosed CD80-targeted therapeutics include primary and metastatic tumors in breast, colon, rectum, lung, oropharynx, hypopharynx, esophagus, stomach, pancreas, liver, gallbladder, bile ducts, small intestine, urinary tract including kidney, bladder and urothelium, female genital tract, cervix, uterus, ovaries, male genital tract, prostate, seminal vesicles, testes, an endocrine gland, thyroid gland, adrenal gland, pituitary gland, skin, bone, soft tissues, blood vessels, brain, nerves, eyes, meninges. Other relevant solid tumors include those having B cell involvement.

III.B. Dose and Administration

The data obtained from cell-based assays and animal studies, usually in rodents, rabbits, dogs, pigs, and/or or primates, can be used in formulating a range of dosage for use in humans. The animal model may also be used to determine the appropriate concentration range and route of administration. Such information may then be used to determine useful doses and routes for administration in humans. Typically, a minimal dose is administered, and the dose is escalated in the absence of dose-limiting cytotoxicity. Determination and adjustment of an effective amount or dose, as well as evaluation of when and how to make such adjustments, are known to those of ordinary skill in the art of medicine.

The dosage of such compounds lies preferably within a range of circulating concentrations that include the ED₅₀ with little or no toxicity. The dosage may vary within this range depending upon the dosage form employed and the route of administration utilized. For any present CD80-targeted therapeutic composition used in the method of the invention, the therapeutically effective amount can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

Efficacious doses of anti-CD80 antibody, such as galiximab, may include weekly doses ranging from 100 mg/m² to 600 mg/m², such as 4 weekly infusions of galiximab at a dose of 125 mg/m², 250 mg/m², 375 mg/m², or 500 mg/m². Anti-CD80 antibodies may also be administered at lower or higher doses, such as doses that are about 90%, or about 80%, or about 70%, or about 60%, or about 50%, or about 40%, or about 30%, or about 20% or about 10%, or less of the above-identified doses for galiximab. For example, effective doses may include weekly doses of 10 mg/m², 12.5 mg/m², 25 mg/m², 37.5 mg/m², or 50 mg/m². In particular, non-fucosylated anti-CD80 antibodies having enhanced ADCC activity, or other enhanced anti-tumor activity, when compared to the same antibody, which is prepared in a manner that does not specifically remove fucose residues, may be effective at a reduced dose. Doses of anti-CD80 antibodies may also include doses that are about 110%, or about 120%, or about 130%, or about 140%, or about 150%, or about 160%, or about 170%, or about 180% or about 190%, or more of the above-identified doses for galiximab.

Pharmaceutical compositions for use in accordance with the invention may be formulated in a conventional manner using one or more physiologically acceptable carriers or excipients. Thus, the compositions and their physiologically acceptable salts and solvates may be formulated for administration by inhalation or insufflation (either through the mouth or the nose) or oral, buccal, parenteral, topical, subcutaneous, intraperitoneal, intraveneous, intrapleural, intraoccular, intraarterial, rectal administration, or within/on implants, e.g., matrices such as collagen fibers or protein polymers, via cell bombardment, in osmotic pumps, grafts comprising appropriately transformed cells, etc. For the treatment of solid tumors, CD80-targeted therapeutics of the invention may be administered directly to the tumor site. For treatment of CNS malignancies, CD80-targeted therapeutics may be administered directly to the CNS, for example, by intrathecal or intraventricular administration. In addition, a variety of techniques are available for promoting transfer of CD80-targeted therapeutics across the blood brain barrier including disruption by surgery or injection, drugs which transiently open adhesion contact between CNS vasculature endothelial cells, and compounds which facilitate translocation through such cells.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Pharmaceutical compositions may also include various buffers (e.g., Tris, acetate, phosphate), solubilizers (e.g., TWEEN®, Polysorbate), carriers such as human serum albumin, preservatives (thimerosol, benzyl alcohol) and anti-oxidants such as ascorbic acid in order to stabilize pharmaceutical activity. The stabilizing agent may be a detergent, such as TWEEN®-20, TWEEN®-80, NP-40 or TRITON-X®-100. EBP may also be incorporated into particulate preparations of polymeric compounds for controlled delivery to a patient over an extended period of time. A more extensive survey of components in pharmaceutical compositions is found in Remington's Pharmaceutical Sciences, 1990, 18th ed., A. R. Gennaro, ed., Mack Publishing, Easton, Pa.

In addition to the formulations described previously, the compounds may also be formulated as a depot preparation. Such long acting formulations may be administered by implantation (for example, subcutaneously or intramuscularly) or by intramuscular injection. Thus, for example, the compounds may be formulated with suitable polymeric or hydrophobic materials (for example, as an emulsion in an acceptable oil) or ion exchange resins, or as sparingly soluble derivatives, for example, as a sparingly soluble salt.

The compositions may, if desired, be presented in a pack or dispenser device that may contain one or more unit dosage forms containing the active ingredient. The pack may for example comprise metal or plastic foil, such as a blister pack. The pack or dispenser device may be accompanied by instructions for administration.

For combination therapies, which include administration of a CD80-targeted therapeutic along with one or more additional therapeutic agents, administration may be performed within any time frame suitable for performance of the intended therapy. For example, a single agent CD80-targeted therapeutic and any additional agents may be administered substantially simultaneously (i.e., as a single formulation or within minutes or hours) or consecutively in any order. For consecutive administration, the single agents are administered with an intervening period of about 10, 8, 6, 4, or 2 months, or with an intervening period of 4, 3, 2 or 1 week(s), or with an intervening period of about 5, 4, 3, 2 or 1 day(s).

When used in combination with one or more additional therapeutic agents, the CD80-targeted therapeutic and the one or more additional agents may be administered or otherwise contacted with cells concurrently or sequentially in either order. The disclosed combination therapies may elicit a synergistic therapeutic effect, i.e., an effect greater than the sum of their individual effects. Measurable therapeutic effects are described herein above. For example, a synergistic therapeutic effect may be an effect of at least about two-fold greater than the therapeutic effect elicited by a single agent, or the sum of the therapeutic effects elicited by the single agents of a given combination, or at least about five-fold greater, or at least about ten-fold greater, or at least about twenty-fold greater, or at least about fifty-fold greater, or at least about one hundred-fold greater. A synergistic therapeutic effect may also be observed as an increase in therapeutic effect of at least 10% compared to the therapeutic effect elicited by a single agent, or the sum of the therapeutic effects elicited by the single agents of a given combination, or at least 20%, or at least 30%, or at least 40%, or at least 50%, or at least 60%, or at least 70%, or at least 80%, or at least 90%, or at least 100%, or more.

For additional guidance regarding formulation, dose, administration regimen, and measurable therapeutic outcomes, see Berkow et al., The Merck Manual of Medical Information, 2000, Merck & Co., Inc., Whitehouse Station, N.J.; Ebadi, CRC Desk Reference of Clinical Pharmacology, 1998, CRC Press, Boca Raton, Fla.; Gennaro, Remington: The Science and Practice of Pharmacy, 2000, Lippincott, Williams & Wilkins, Philadelphia, Pa.; Katzung, Basic & Clinical Pharmacology, 2001, Lange Medical Books/McGraw-Hill Medical Pub. Div., New York; Hardman et al., Goodman & Gilman's the Pharmacological Basis of Therapeutics, 2001, The McGraw-Hill Companies, Columbus, Ohio; Speight & Holford, Avery's Drug Treatment: A Guide to the Properties, Choices, Therapeutic Use and Economic Value of Drugs in Disease Management, 1997, Lippincott, Williams, & Wilkins, Philadelphia, Pa.

II.C. Combination Therapies

The CD80-targeted therapeutics as described herein may be used in combination with other therapeutic agents or other therapies (e.g., surgical excision, radiation, etc.) to thereby elicit an enhanced therapeutic effect and/or to reduce hepatocytotoxicity of some therapeutic agents. When used in combination with one or more additional therapeutic agents, the CD80-targeted therapeutics and the one or more additional agents may be administered or otherwise contacted with cells concurrently or sequentially in either order. The disclosed combination therapies may elicit a synergistic therapeutic effect, i.e., an effect greater than the sum of their individual effects. Measurable therapeutic effects are described herein above. For example, a synergistic therapeutic effect may be an effect of at least about two-fold greater than the therapeutic effect elicited by a single agent, or the sum of the therapeutic effects elicited by the single agents of a given combination, or at least about five-fold greater, or at least about ten-fold greater, or at least about twenty-fold greater, or at least about fifty-fold greater, or at least about one hundred-fold greater. A synergistic therapeutic effect may also be observed as an increase in therapeutic effect of at least 10% compared to the therapeutic effect elicited by a single agent, or the sum of the therapeutic effects elicited by the single agents of a given combination, or at least 20%, or at least 30%, or at least 40%, or at least 50%, or at least 60%, or at least 70%, or at least 80%, or at least 90%, or at least 100%, or more.

For the treatment of malignancies, representative agents useful for combination therapy include cytotoxins, radioisotopes, chemotherapeutic agents, immunomodulatory or immunoregulatory agents, anti-angiogenic agents, anti-proliferative agents, pro-apoptotic agents, cytostatic and cytolytic enzymes (e.g., RNAses), enzyme inhibitors (e.g., proteasome inhibitors), and tumor vaccines. Additional agents include a therapeutic nucleic acid, such as a gene encoding an immunomodulatory agent, an anti-angiogenic agent, an anti-proliferative agent, or a pro-apoptotic agent. These drug descriptors are not mutually exclusive, and thus a therapeutic agent may be described using one or more of the above-noted terms. CD80-targeted therapeutics of the invention may also be used in combination with agents that deplete regulatory T cells, or which block, inhibit, or otherwise downregulate regulatory T cell functions.

The term cytotoxin generally refers to an agent that inhibits or prevents the function of cells and/or results in destruction of cells. Representative cytotoxins include antibiotics, inhibitors of tubulin polymerization, alkylating agents that bind to and disrupt DNA, and agents that disrupt protein synthesis or the function of essential cellular proteins such as protein kinases, phosphatases, topoisomerases, enzymes, and cyclins. Representative cytotoxins include, but are not limited to, doxorubicin, daunorubicin, idarubicin, aclarubicin, zorubicin, mitoxantrone, epirubicin, carubicin, nogalamycin, menogaril, pitarubicin, valrubicin, cytarabine, gemcitabine, trifluridine, ancitabine, enocitabine, azacitidine, doxifluridine, pentostatin, broxuridine, capecitabine, cladribine, decitabine, floxuridine, fludarabine, gougerotin, puromycin, tegafur, tiazofurin, adriamycin, cisplatin, carboplatin, cyclophosphamide, dacarbazine, vinblastine, vincristine, mitoxantrone, bleomycin, mechlorethamine, prednisone, procarbazine, methotrexate, fluorouracils, etoposide, taxol, taxol analogs, platins such as cis-platin and carbo-platin, mitomycin, thiotepa, taxanes, vincristine, daunorubicin, epirubicin, actinomycin, authramycin, azaserines, bleomycins, tamoxifen, idarubicin, dolastatins/auristatins, hemiasterlins, esperamicins and maytansinoids.

Radioisotopes suitable for radiotherapy include but are not limited to α-emitters, β-emitters, and auger electrons. These radioisotopes are typically conjugated to a targeting antibody for delivery to disease cells. For example, radiolabeled antibodies can include a radioisotope such as ¹⁸fluorine, ⁶⁴copper, ⁶⁵copper, ⁶⁷gallium, ⁶⁸gallium, ⁷⁷bromine, ^{80m}bromine, ⁹⁵ruthenium, ⁹⁷ruthenium, ¹⁰³ruthenium, ¹⁰⁵ruthenium, ^{99m}technetium, ¹⁰⁷mercury, ²⁰³mercury, ¹²³iodine, ¹²⁴iodine, ¹²⁵iodine, ¹²⁶iodine, ¹³¹iodine, ¹³³iodine, ¹¹¹indium, ¹¹³indium, ^{99m}rhenium, ¹⁰⁵rhenium, ¹⁰¹rhenium, ¹⁸⁶rhenium, ¹⁸⁸rhenium, ¹²¹mtellurium, ⁹⁹technetium, ^{122m}tellurium, ^{125m}tellurium, ¹⁶⁵thulium, ¹⁶⁷thulium, ¹⁶⁸thulium, ⁹⁰yttrium, alpha emitters, such as ²¹³bismuth, ²¹³lead, and ²²⁵actinium, and nitride or oxide forms derived there from.

Immunomodulatory or immunoregulatory agents are compositions that elicit an immune response, including humoral immune responses (e.g., production of antigen-specific antibodies) and cell-mediated immune responses (e.g., lymphocyte proliferation). Representative immunomodulatory agents include cytokines, xanthines, interleukins, interferons, and growth factors (e.g., TNF, CSF, GM-CSF and G-CSF), and hormones such as estrogens (diethylstilbestrol, estradiol), androgens (testosterone, HALOTESTIN® (fluoxymesterone)), progestins (MEGACE® (megestrol acetate), PROVERA® (medroxyprogesterone acetate)), and corticosteroids (prednisone, dexamethasone, hydrocortisone).

Immunomodulatory agents useful in the invention also include anti-hormones that block hormone action on tumors and immunosuppressive agents that suppress cytokine production, down-regulate self-antigen expression, or mask MHC antigens. Representative anti-hormones include anti-estrogens including, for example, tamoxifen, raloxifene, aromatase inhibiting 4(5)-imidazoles, 4-hydroxytamoxifen, trioxifene, keoxifene, LY 117018, onapnstone, and toremifene; and anti-androgens such as flutamide, nilutamide, bicalutamide, leuprolide, and goserelin; and anti-adrenal agents. Representative immunosuppressive agents include 2-amino-6-aryl-5-substituted pyrimidines, azathioprine, cyclophosphamide, bromocryptine, danazol, dapsone, glutaraldehyde, anti-idiotypic antibodies for MHC antigens and MHC fragments, cyclosporin A, steroids such as glucocorticosteroids, cytokine or cytokine receptor antagonists (e.g., anti-interferon antibodies, anti-IL10 antibodies, anti-TNFα antibodies, anti-IL2 antibodies), streptokinase, TGFβ, rapamycin, T-cell receptor, T-cell receptor fragments, and T cell receptor antibodies.

Additional drugs useful in the invention include anti-angiogenic agents that inhibit blood vessel formation, for example, farnesyltransferase inhibitors, COX-2 inhibitors, VEGF inhibitors, bFGF inhibitors, steroid sulphatase inhibitors (e.g., 2-methoxyoestradiol bis-sulphamate (2-MeOE2bisMATE)), interleukin-24, thrombospondin, metallospondin proteins, class I interferons, interleukin 12, protamine, angiostatin, laminin, endostatin, and prolactin fragments.

Anti-proliferative agents and pro-apoptotic agents include activators of PPAR-gamma (e.g., cyclopentenone prostaglandins (cyPGs)), retinoids, triterpinoids (e.g., cycloartane, lupane, ursane, oleanane, friedelane, dammarane, cucurbitacin, and limonoid triterpenoids), inhibitors of EGF receptor (e.g., HER4), rampamycin, CALCITRIOL® (1,25-dihydroxycholecalciferol (vitamin D)), aromatase inhibitors (FEMARA® (letrozone)), telomerase inhibitors, iron chelators (e.g., 3-aminopyridine-2-carboxaldehyde thiosemicarbazone (Triapine)), apoptin (viral protein 3—VP3 from chicken aneamia virus), inhibitors of Bcl-2 and Bcl-X(L), TNF-alpha, FAS ligand, TNF-related apoptosis-inducing ligand (TRAIL/Apo2L), activators of TNF-alpha/FAS ligand/TNF-related apoptosis-inducing ligand (TRAIL/Apo2L) signaling, and inhibitors of PI3K-Akt survival pathway signaling (e.g., UCN-01 and geldanamycin).

Additional agents that may be used in combination with CD80 antagonists include agents that block interaction of B lymphocyte stimulator (BLyS) with one or more of its receptors, B cell activating factor receptor (BAFF-R), transmembrane activator and calcium modulator and cyclophilin ligand interactor (TACI), or B-cell maturation antibody (BCMA). For example, useful agents include antibodies that specifically bind the BLyS ligand or antibodies that specifically bind one or more of its receptors, including any of the antibody types described herein. Small molecule inhibitors of the interaction of BLyS with one or more of its receptors may also be used.

CD80-targeted therapeutics of the invention may also be used in combination with other anti-cancer therapeutic antibodies and antibody/drug conjugates. Representative antibodies, which may be used in unlabeled/unconjugated form or as an antibody/drug conjugate, include anti-CD19 antibodies, anti-CD20 antibodies (e.g., RITUXAN®, ZEVALIN®, BEXXAR®), anti-CD22 antibodies, anti-CD33 antibodies (e.g., MYLOTARG®), anti-CD33 antibody/drug conjugates, anti-Lewis Y antibodies (e.g., Hu3S193, Mthu3S193, AGmthu3S193), anti-HER-2 antibodies (e.g., HERCEPTIN® (trastuzumab), MDX-210, OMNITARG® (pertuzumab, rhuMAb 2C4)), anti-CD52 antibodies (e.g., CAMPATH®), anti-EGFR antibodies (e.g., ERBITUX® (cetuximab), ABX-EGF (panitumumab)), anti-VEGF antibodies (e.g., AVASTIN® (bevacizumab)), anti-DNA/histone complex antibodies (e.g., ch-TNT-1/b), anti-CEA antibodies (e.g., CEA-Cide, YMB-1003) hLM609, anti-CD47 antibodies (e.g., 6H9), anti-VEGFR2 (or kinase insert domain-containing receptor, KDR) antibodies (e.g., IMC-1C11), anti-Ep-CAM antibodies (e.g., ING-1), anti-FAP antibodies (e.g., sibrotuzumab), anti-DR4 antibodies (e.g., TRAIL-R), anti-progesterone receptor antibodies (e.g., 2C5), anti-CA19.9 antibodies (e.g., GIVAREX®) and anti-fibrin antibodies (e.g., MH-1).

CD80-targeted therapeutics of the invention may also be used in combination with systemic anti-cancer drugs, such as epithilones (BMS-247550, Epo-906), reformulations of taxanes (Abraxane, Xyotax), microtubulin inhibitors (MST-997, TTI-237), etc.

In other combination therapies, CD80-targeted therapeutics may be administered together with one or more combinations of chemotherapeutic agents, for example, alkylating agents such as thiotepa and cyclosphosphamide; alkyl sulfonates such as busulfan, improsulfan and piposulfan; aziidines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chiorambucil, chlornaphazine, cholophosphamide, estramustine, ifosfamide, mechiorethamine, mechiorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-EU; androgens such as calusterone, dromostanolone propionate, epitiostanol, mepitiostane, testolactone; anti-adrenal such as aminoglutethimide, mitotane, trilostane; folic acid replenisher such as frolinic acid; aceglatone; aldophosphamide glycoside; arninolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; razoxane; sizofuran; spirogermanium; tenuazonic acid; triaziquone; 2,2′,2′-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel (TAXOL®, Bristol-Myers Squibb Oncology of Princeton, N.J.) and doxetaxel (TAXOTERE®, Rhone-Poulenc Rorer of Antony, France); chiorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylornithine (DMFO); retinoic acid; esperarnicins; capecitabine and combinations of therapeutic agents such as ABVD (adriamycin, bleomycin, vincristine, dacarbazine).

For the treatment of Hodgkin's Disease, CD80-targeted therapeutics of the invention may be used in combination with antibodies that bind to antigens expressed on Hodgkin Reed Sternberg (HRS) cells or cells of the infiltrate surrounding HRS cells. Such antigens that are useful for targeting of HRS cells include CD30, CD40, RANK, TRAIL, Notch, LMP, IL-13, CD20, CD52, and CCR4. Other useful agents for combination therapies for treating Hodgkin's Disease include proteosome inhibitors (e.g., Bortezomib (PS-341; Millennium Pharmaceuticals) and MG-132 (Tokyo, Metropolitan Institute of Medical Science)), histone deacytylase inhibitors (e.g., depsipeptide (FK228; Gloucester Pharmaceuticals) and suberoylanilide hydroxamic acid (SAHA; Aton Pharma)), and small molecules and peptides that may be used to induce apoptosis of HRS cells, including triterpenoids, such as CDD (RTA401, Reata Discovery) and 17-allylamino-17-demethoxy-gledanamycin (see, e.g., Kamal et al., Trends. Mol. Med., 2004, 10, 283-290), N-acetyl-leucinyl-leucynil-norleucynal, N-acetyl-leucinyl leucynil-methional, carbobenzoxyl-leucinyl-leucynil-norvalinal, carbobenzoxyl-leucinyl-leucynil-leucynal, β-lactone, bactacystine, boronic acid peptides, ubiquitin ligase inhibitors, cyclosporin A, and deoxyspergualin.

CD80-targeted therapeutics of the invention may also be used in combination with agents that promote anti-tumor immunity, for example, tumor vaccines. Many such vaccines are known in the art, including vaccines that incorporate tumor-specific cytotoxic epitopes, as well as helper epitopes. Additional agents may prevent the induction of CD8⁺ cytolytic T lymphocyte anergy.

Still further, CD80-targeted therapeutics of the invention may be used in combination with agents that enhance immune activity, for example, anti-CTLA4 antibodies, other agents that block CTLA4, anti-PD1 antibodies, and additional agents that release inhibitory controls on T cell activation and proliferation.

Throughout this application, various publications, patents and published patent applications are referred to by an identifying citation. The disclosures of these publications, patents and published patent specifications referenced in this application are hereby incorporated by reference into the present disclosure to more fully describe the state of the art to which this invention pertains.

The following examples have been included to illustrate modes of the invention. Certain aspects of the following examples are described in terms of techniques and procedures found or contemplated by the present co-inventors to work well in the practice of the invention. In light of the present disclosure and the general level of skill in the art, those of skill will appreciate that the following examples are intended to be exemplary only and that numerous changes, modifications, and alterations may be employed without departing from the scope of the invention.

### EXAMPLES

### Example 1

### Galiximab for Treatment of Relapsed or Refractory Hodgkin's Lymphoma

Adult patients (at least 18 years old) with histologically confirmed classical Hodgkin's lymphoma are selected for treatment with the anti-CD80 antibody, galiximab. Galiximab is an IgG1 lambda, anti-CD80 monoclonal antibody developed using PRIMATIZED® antibody technology to decrease immunogenicity. The variable regions are of cynomologous macaque origin, and the constant regions are of human origin. Galiximab is formulated for intravenous injection as a sterile product in 10 mmol/L sodium citrate, containing 150 mmol/L sodium chloride and 0.02% polysorbate 80 at pH 6.5.

Patients have a measurable disease (e.g., at least one lesion ≧10 mm) and adequate hematologic, renal and hepatic function. Patients are treated with galiximab for a period of one month. The patients are pre-medicated with 50 mg of diphenhydramine administered by intravenous infusion (IV) using an infusion pump and a 0.22 micron low-protein binding filter or by mouth and 650 mg of orally administered acetaminophen prior to intravenous infusions of galiximab. Patients receive 500 mg/m² of galiximab once weekly for 4 weeks. Galiximab is administered in an outpatient setting over a 1-hour period. Patients are monitored for at least one hour following completion of galiximab infusion. Antibody doses are diluted in 150 mL-250 mL of normal saline. Galiximab infusions are withheld if any infusion reaction (e.g., fever, chills, dyspnea) ≧grade 1 occurs. After resolution of the symptoms, the infusion may be restarted at half the previous rate. Galiximab infusion is stopped if the patient experiences a second infusion-related adverse event, and not restarted the same day.

After the administration of the induction therapy as described above, patients receive extended galiximab therapy on a monthly basis until disease progression. Patients receive 500 mg/m² doses of galiximab every four weeks over 60 minutes using an infusion pump and a 0.22 micron low-protein binding filter. The infusion time is extended if the patient experiences infusion-related toxicity. Patients who respond to galiximab are allowed to continue treatment. Their progress is examined at week eight and then after every 3 galiximab treatments (i.e., every 12 weeks) until disease progression. All patients who withdraw from the study early or who are alive at the end of the 48-month study period are observed at 6-month intervals for continuation of response, initiation of other lymphoma therapy, survival status or cause of death. Galiximab treatment results in changes within the tumor microenvironment that promote tumor immunity, including enhancement of adaptive immune responses, for example, by inhibition of regulatory T cell suppression, a decrease in immunoregulatory cells and/or inflammatory cells in the tumor microenvironment, down-regulation of cytokines and other factors that support tumor progression, a decrease in tumor cell growth and/or migration, a decrease in tumor vascularization, etc.

Evaluation of disease is performed by comprehensive scans (computed tomography, magnetic resonance imaging, and x-rays) and physical examination at baseline (study entry), 1 month after completion of galiximab treatment (day 50), every 3 months thereafter for years 1 and 2, and every 6 months for years 3 and 4. Response to treatment is analyzed using standard outcome measures for clinical trials (complete response, unconfirmed complete response, partial response, stable disease and progressive disease) as defined by the International Workshop Response Criteria for Non-Hodgkin's lymphoma. Relevant end points include overall response rate, complete remission rate, unconfirmed complete remission rate, partial remission rate, duration of response and time to progression. Additional relevant indices of efficacy include changes in the tumor microenvironment as assessed in Example 3.

### Example 2

### Pharmacokinetic Analysis of Galiximab

Serum and/or biopsy samples are obtained before infusion, and within 10 minutes of the completion of infusions (days 1, 8, 15 and 22), prior to galiximab infusion at week eight and thereafter every 12 weeks for as long as the patient remains on the study. Pharmacokinetic analysis analyses include galiximab concentrations in serum, tumor mass, tumor nodules, and/or tumor draining lymph nodes; maximum observed concentration (Cmax); time to maximum observed concentration; half-life; and the area under the concentration time curve (AUC). Data are analyzed using a non-compartmental linear regression method to determine the antibody half-life using data from all samples collected after study day 1 that contain galiximab concentrations exceeding the lower limit of quantitation for the assay (250 ng/mL). AUC is calculated using the linear/logarithmic trapezoidal method and determined with time extrapolated out to infinity.

### Example 3

### Effect of Galiximab on Immune Functions in the Tumor Microenvironment

In patients receiving galiximab therapy, serum and/or biopsies are obtained at the following time points: prior to treatment on day 1, in week 4 (completion of induction), in week 8, and every 12 weeks thereafter for the duration that the patient remains on study. Biopsies may be obtained from a tumor mass, tumor nodules, and/or tumor draining lymph nodes. Immune cell functions in the samples are assayed according to methods known in the art. Cytokine levels are determined using antibodies that specifically bind to the cytokine(s) of interest. Regulatory T cells, Th2 cells, and macrophages are quantified using flow cytometric analyses using appropriate molecular markers as known in the art and described herein. For analysis of the tumor microenvironment, malignant cells are first depleted from the sample using an antibody that specifically binds a tumor-associated antigen. One skilled in the art can readily identify an appropriate tumor-associated antigen for the malignancy being treated. Regulatory T cell content is assessed by identifying a population of CD4⁺, CD25^{hi} cells, optionally in combination with one or more of cytotoxic T lymphocyte-associated antigen 4 (CTLA4), glucocorticoid-induced tumor necrosis factor receptor family-related gene (GITR), CC chemokine receptor 4 (CCR4), forkhead box p3 (FOXP3), CC chemokine receptor 6 (CCR6), CD30, CD62L¹, CD45RB^{lo}, CD45RO^{hi}, and/or CD45RA⁻. Th2 cells may be identified TCR⁺ (T cell receptor) CD4⁺, CD25⁻ cells. Macrophages are quantified using biomarkers including TLR5, FCGR1A, SEPT10, LGMN, and/or C3AR1. Macrophages may also be quantified using biomarkers to calculate lymphocyte associated macrophage (LAM) content, for example, as described by Farinha et al., Blood, 2005, 106(16):2169-2174. Regulatory T cell proliferation is assayed by culturing of cells in the presence of a vital dye, such as carboxyfluorescein diacetate (CFSE).

### Example 4

### Effect of Galiximab on T Cell Activation

The effect of galiximab on the activation of normal human peripheral blood T lymphocytes was determined by culturing CD4+CD25− T cells with either SB lymphoma cells or with mature dendritic cells obtained from an independent donor. In both cases, the stimulus for T cell activation was provided by allogeneic major histocompatibility complex (MHC) proteins expressed by either the SB lymphoma cells or the mature dendritic cells. The extent of MHC mismatch was not further characterized.

Peripheral blood mononuclear cells (PBMC) were isolated by density centrifugation using a standard Ficoll-paque procedure. CD4+CD25− T lymphocytes were negatively selected from the PBMC population by indirect magnetic labeling of all cells lacking CD4 expression and magnetic cell separation using the CD4+ T cell isolation kit (Meltenyi Biotec of Aubern, Calif.). Depletion of cells expressing CD8, CD14, CD16, CD19, CD36, CD56, CD123, TCR gamma/delta and CD235a (glycophorin A) resulted in a population of CD4+ T lymphocytes that was greater than 95% positive for CD4 expression. This population of CD4+ T cells was further depleted of CD25 expressing cells by negative selection using CD25 Microbeads II. (Meltenyi Biotec of Aubern, Calif.). All cellular isolations were characterized by flow cytometry to ensure consistency of the enriched cell populations. CD4+CD25− T cells were subsequently labeled with the cell permeable fluorescent dye carboxy fluoroscein succinimidyl ester (CFSE). Mature dendritic cells were generated by culturing PBMC overnight in the presence of 2 ug/ml lipopolysaccharide. The cells were subsequently washed five times and subject to external gamma irradiation (2000 rad) to render the cells non-proliferative. CFSE-labeled CD4+CD25− T cells were cultured with either SB lymphoma cells or mature dendritic cells for five days. Varying concentrations of galiximab were included in the cultures. In addition, the effect of a blocking antibody to CD86 and CTLA4-Ig were also investigated for purposes of comparison. CD4+CD25− T cell proliferation was measured by flow cytometric quantitation of the intensity of the CFSE fluorescent dye, which is distributed between daughter cells upon cell division, resulting in a progressive diminution in cellular fluorescence intensity with increasing cycles of cell division.

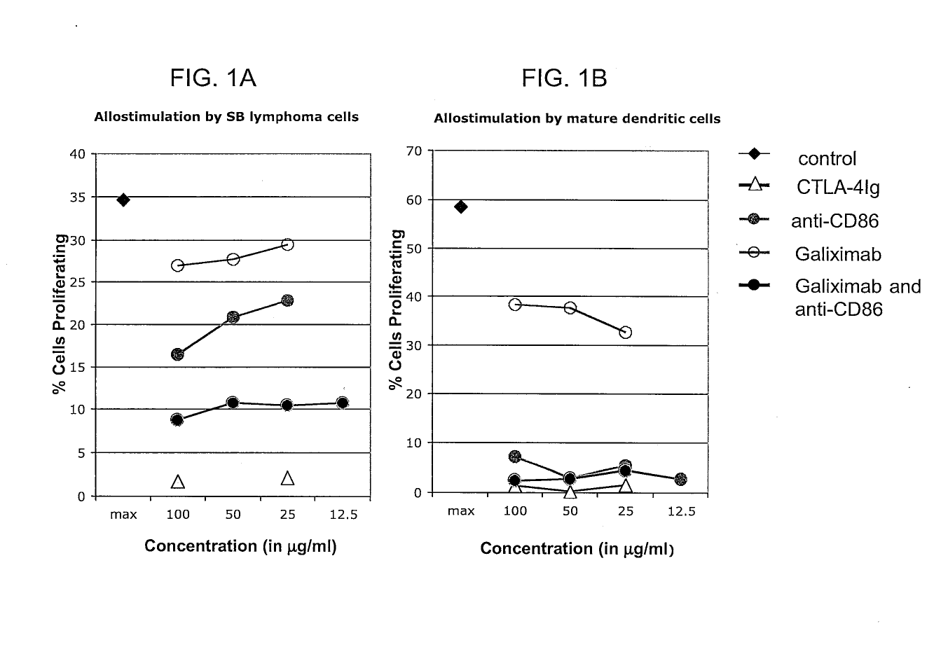

The proliferation of CD4+CD25− T cells induced by either SB lymphoma cells or by allogeneic dendritic cells was significantly inhibited upon inclusion of galiximab in the cell culture (FIGS. 1A-1B). Inhibition of T cell proliferation by galiximab was consistently less than inhibition mediated by either anti-CD86 or CTLA4-Ig antibodies. This is consistent with the relative contribution of CD80 versus CD86 as costimulatory molecules supporting T cell activation. These results demonstrate that within the context of a strong T cell activation signal (i.e. an allogeneic stimulation), galiximab partially inhibits activation of naïve CD4+CD25− T lymphocytes, as manifest by inhibition of T cell proliferation. To the extent that T lymphocytes contribute to establishing the unique composition of cells present in a tumor microenvironment, these results indicate that galiximab is capable of modulating T cell function and thereby alter the contribution by T lymphocytes in defining a tumor microenvironment.

### Example 5

### Effect of Galiximab on Cytokine Production by Activated T Cells

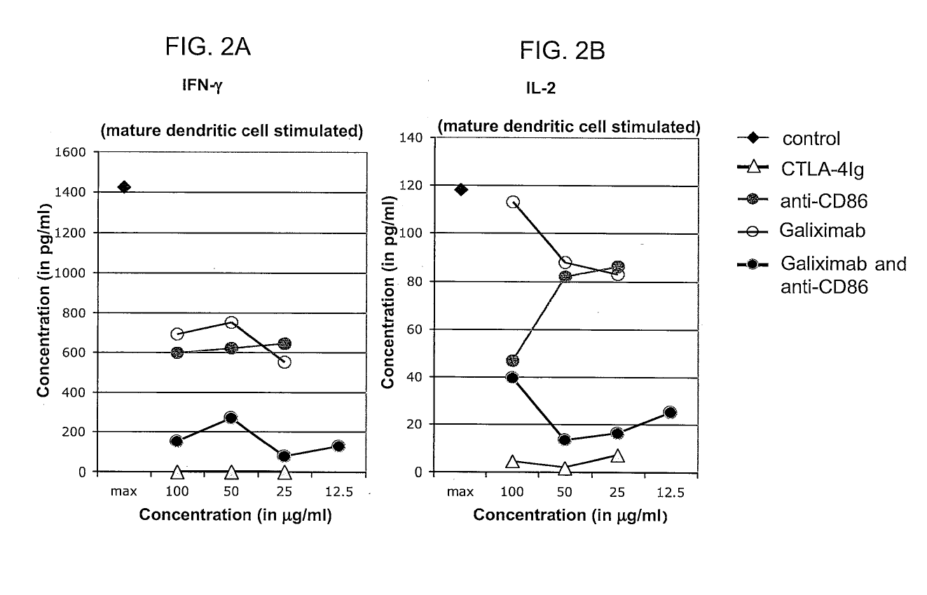

CD4+CD25− T cells were isolated and cultured as described in Example 4. The levels of interferon-gamma and interleukin 2 produced upon T cell activation were measured using a multiplexed cytometric bead array assay (BD Biosciences of San Jose, Calif.). Briefly, culture supernatants were collected on day 3 from cultures established as described in example 4 and assayed at multiple dilutions. Concentrations were determined through extrapolation from a concurrently generated standard curve (as per manufacturer's instructions). The production of both interferon-gamma and interleukin-2 induced by culture of CD4+CD25− T lymphocytes with mature, allogeneic dendritic cells was significantly inhibited by galiximab (FIGS. 2A-2B). Galiximab and anti-CD86 each inhibited interferon-gamma production by approximately 50% (FIG. 2A). The combination of galiximab and anti-CD86 resulted in near complete inhibition of interferon-gamma production (FIG. 2A). CTLA4-Ig also exhibited essentially complete inhibition of interferon-gamma production, consistent with the additive effect of galiximab and anti-CD86 given that CTLA4-Ig binds to and blocks both CD80 and CD86 (FIG. 2A). Production of interleukin-2 exhibited similar differential inhibition by galiximab, anti-CD 86, CTLA4-Ig and galiximab plus anti-CD86 (FIG. 2B). These results demonstrate that within the context of a strong T cell activation signal (i.e. an allogeneic stimulation), galiximab partially inhibits activation of naïve CD4+CD25− T lymphocytes, as manifest by inhibition of interferon-gamma and interleukin-2 production. To the extent that T lymphocytes contribute to establishing the unique composition of cytokines present in a tumor microenvironment, these results indicate that galiximab is capable of modulating T cell function and thereby alter the contribution by T lymphocytes in defining a tumor microenvironment.

### Example 6

### Effect of Galiximab on Regulatory T Cell Development

The effect of galiximab on the generation of regulatory T cells was investigated in an ex vivo co-culture system in which CD4+CD25+ T lymphocytes were cultured with allogeneic SB lymphoma cells in a 3 day culture. Briefly, PBMC were isolated as described in example 4 and CD4+ cells were further purified by negative selection as described in example 4. CD4+CD25+ cells were further enriched by positive selection using CD25 Microbeads II. (Meltenyi Biotec of Aubern, Calif.). Regulatory T cells are measured by flow cytometry as the percentage of CD4+CD45RA+CD127− cells that express high levels of CD25, i.e., CD25hi. This cell population was validated to represent FOXP3-positive cells, which defines a regulatory T cell subset.

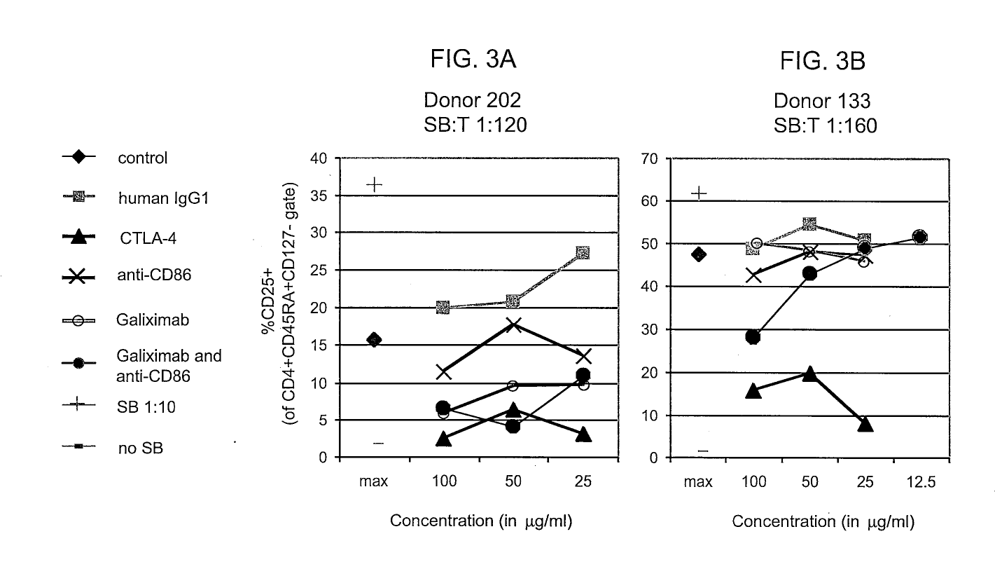

Culture of CD4+CD25+ T cells with SB lymphoma cells at an SB:T ratio of 1:10 resulted in a maximal induction of regulatory T cells to a level representing approximately 37% and 62% (i.e. CD25hi) of CD4+CD45RA+CD127− T cells in assays employing PBMCs from two different donors (FIGS. 3A and 3B). Use of SB:T ratios of 1:120 and 1:160 resulted in an induction of approximately 16% and 48% regulatory T cells, respectively (FIGS. 3A and 3B). In both cases, T cells cultured alone resulted in less than 5% regulatory T cells (FIGS. 3A and 3B). One donor (202) exhibited significant inhibition of regulatory T cell induction by galiximab relative to an isotype-matched human IgG1 control antibody (FIG. 3A). In this case, galiximab was more potent than anti-CD86, exhibiting a level of inhibition of regulatory T cell induction that was more similar to that observed with CTLA4-Ig or with galiximab plus anti-CD86. Another donor exhibited inhibition of regulatory T cell induction by CTLA4-Ig but not by galiximab (FIG. 3B). These results indicate that galiximab is capable of inhibiting the induction of regulatory T cells.

### Example 7

### Effect of Galiximab on Cytokine Production in a Tumor Microenvironment

To further investigate the potential effect of galiximab within the context of the tumor microenvironment, a co-culture assay was established to model the contribution of multiple cell types that to varying extents contribute to establishing a complex tumor microenvironment.

PBMC were isolated by density centrifugation using a standard Ficoll-paque procedure. Specific cellular subsets, consisting of either CD4+ T lymphocytes or CD14+ monocytes, were negatively selected from the PBMC population by indirect magnetic labeling of all non CD4+ or CD14+ cells and magnetic cell separation. CD4+ T lymphocytes were isolated to greater than 95% CD4 homogeneity by depletion of cells that express CD8, CD14, CD16, CD19, CD36, CD56, CD123, TCR gamma/delta and CD235a (glycophorin A) the CD4+ T cell isolation kit (Meltenyi Biotec, Aubern, Calif.). CD14+ monocytes were isolated to greater than 90% CD14 homogeneity by depletion of cells that express CD3, CD7, CD16, CD19, CD56, CD123, and CD235a (Glycophorin A) the monocyte isolation kit (Meltenyi Biotec of Aubern, Calif.). All cellular isolations were characterized by flow cytometry to ensure consistency of the enriched cell populations. Cells were cultured in RPMI-1640 culture medium (Invitrogen of Carlsbad, Calif.) containing 10% FBS in 96-well flat-bottom microtiter plates using purified CD4+ T lymphocytes and CD14+ monocytes at 200,000 cells per well each. The Raji lymphoma cell line was cultured at 20,000 cells per well. Cultures were maintained for 4 days, at which time culture supernatants were harvested. A multiplexed cytometric bead array (BD Biosciences of San Jose, Calif.) was used to measure the concentration of interleukin-12 (p70), TN F-alpha, interleukin-10, interleukin-6, interleukin-1 beta, and interleukin-8 present in culture supernatants. Purified human serum myeloma IgG1, kappa (Southern Biotech of Birmingham, Ala.) was used as an isotype control for galiximab. CTLA4-Ig purified from an engineered CHO production cell line was used as a control to compare complete (i.e. CD80 and CD86) blockade with specific blockade of CD80 mediated by galiximab. Lipopolysaccharide (Sigma of St. Louis, Mo.) was used as a positive control for activation of peripheral blood monocytes.

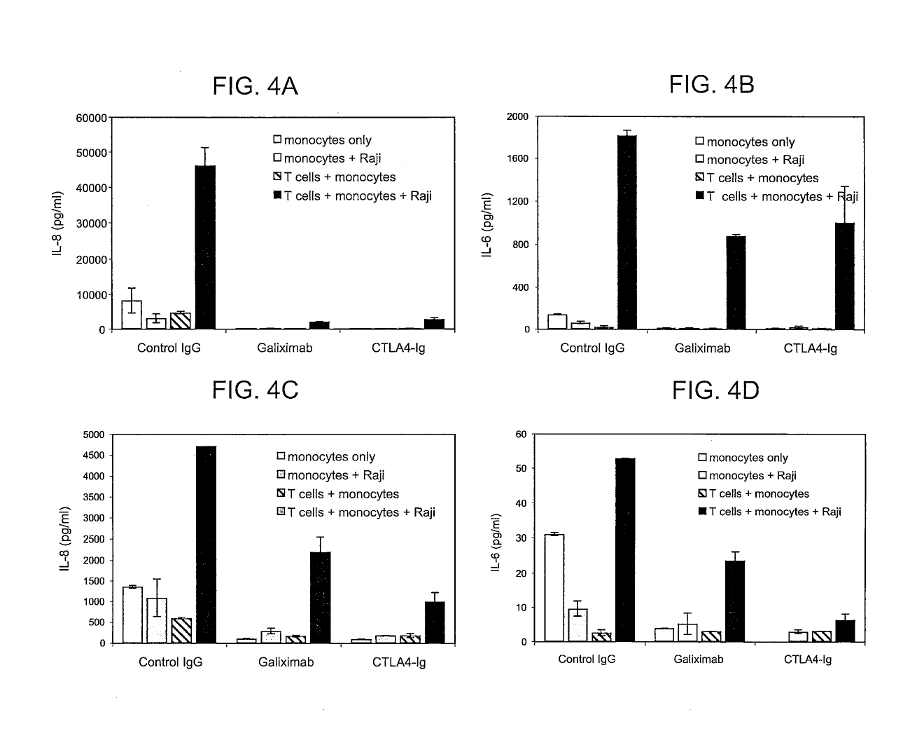

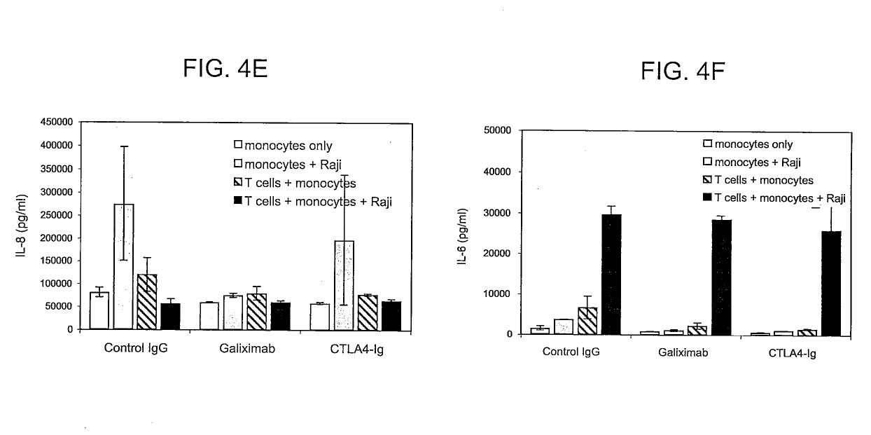

As shown in FIGS. 4A-4D, CD14+ monocytes cultured in isolation exhibited spontaneous or constitutive production of interleukin-8 and interleukin-6. Co-culture with either CD4+ T cells or Raji cells resulted in reduced production of interleukin-8 and interleukin-6 (FIGS. 4A-4D). Galiximab as well as CTLA4-Ig, inhibited production of interleukin-8 and interleukin-6 by either CD14+ monocytes alone or CD14+ monocytes cultured with either CD4+ T cells (1:1 ratio) or Raji cells (10:1 ratio) (FIGS. 4A-4D). The three-way co-culture of CD4+ T cells, CD14+ monocytes and Raji lymphoma cells (10:10:1 ratio) clearly demonstrated a distinct biologic activity that was dependent on the presence of all three cell types, as demonstrated by the marked, synergistic enhancement in both interleukin-8 and interleukin-6 production upon co-culture of all three cell types compared to cultures consisting of any single cell type or combination of two cell types. Galiximab significantly inhibited the enhanced production of both interleukin-8 and interleukin-6 in the three-way co-culture (FIGS. 4A-4D). Results from one donor (donor A) showed galiximab to be equally potent as CTLA4-Ig in inhibiting interleukin-8 and interleukin-6 in the three-way co-culture (FIGS. 4A-4B). Results from a second donor (donor B) showed CTLA4-Ig to exhibit greater potency. In donor B, galiximab inhibited both interleukin-8 and interleukin-6 production by at least 50%. In donor B, galiximab exhibited similar potency as compared to CTLA4-Ig in inhibiting cytokine production by either CD14+ monocytes alone or in combination with either CD4+ T cells or Raji cells (FIGS. 4C-4D). The enhanced potency of CTLA4-Ig in the case of donor B was therefore manifest only under the three-way co-culture conditions in which interleukin-8 and interleukin-6 production was significantly augmented.

Variation was observed with cells obtained from one additional donor (donor C) that were analyzed in the co-culture assay system (FIGS. 4E-4F). In this case, neither galiximab or CTLA4-Ig had any effect on interleukin-8 and interleukin-6 production. However, the level of spontaneous interleukin-8 and interleukin-6 produced by CD14+ monocytes was at least an order of magnitude greater than that observed in assays performed with cells derived from donor A or donor B (FIGS. 4A-4D), as summarized in table 1. In addition, three-way co-culture employing cells from donor C did not result in synergistically augmented production of interleukin-8, as was observed with cells from donors A and B, although synergistic enhancement of interleukin-6 production was observed. These results demonstrate that galiximab can significantly impact the spectrum of cytokines expressed within the tumor microenvironment that directly and indirectly support tumor progression.

TABLE 1
Spontaneous cytokine production by CD14+ monocytes

| donor | Interleukin-8 (pg/ml) | Interleukin-6 (pg/ml) |
|---|---|---|
| A | 8244 +/− 3548 | 144 +/− 3 |
| B | 1362 +/− 34 | 31 +/− 0 |
| C | 82094 +/− 10983 | 1633 +/− 530 |

1. A method of treating a subject having a malignancy which comprises a tumor microenvironment of malignant and non-malignant cells wherein regulatory T cell function and/or non-malignant CD80-expressing cells contributes to or exacerbates the malignancy, comprising administering to the subject a therapeutically effective amount of an anti-CD80 antibody or a CD80-binding fragment thereof in an amount sufficient to modulate the function of the non-malignant cells within the tumor microenvironment that promote tumor growth and survival, to thereby elicit immunodulatory effects.

2. The method of claim 1, wherein the malignancy is a hematologic malignancy or a non-hematologic malignancy.

3. The method of claim 2, wherein the malignancy is a hematologic malignancy selected from the group consisting of a lymphoma, a B cell lymphoma, and Hodgkin's disease.

4-6. (canceled)

7. The method of claim 2, wherein the malignancy is a non-hematologic malignancy.

8. The method of claim 7, wherein the non-hematologic malignancy is a cancer of the breast, colon, rectum, lung, oropharynx, hypopharynx, esophagus, stomach, pancreas, liver, gallbladder, bile ducts, small intestine, urinary tract including kidney, bladder and urothelium, female genital tract, cervix, uterus, ovaries, male genital tract, prostate, seminal vesicles, testes, an endocrine gland, thyroid gland, adrenal gland, pituitary gland, skin, bone, soft tissues, blood vessels, brain, nerves, eyes, meninges.

9. (canceled)

10. The method according to claim 1, wherein the anti-CD80 antibody or CD80-binding fragment thereof is a chimeric, humanized, or human antibody.

11. The method according to claim 1, wherein the anti-CD80 antibody or CD80 binding fragment thereof is selected from the group consisting of an anti-CD80 antibody or fragment thereof which binds a CD80 epitope bound by the antibody produced by ATCC Deposit No. HB-12119, an anti-CD80 antibody or fragment thereof which competes for binding to CD80 with the antibody produced by ATCC Deposit No. HB-12119, an anti-CD80 antibody or a CD80-binding fragment thereof which comprises variable regions derived from variable regions of the antibody produced ATCC Deposit No. HB-12119, an anti-CD80 antibody or a CD80-binding fragment thereof which comprises variable regions of the antibody produced by ATCC Deposit No. HB-12119, an anti-CD80 antibody or a CD80-binding fragment thereof which comprises complementarily determining regions (CDRs) of the antibody produced by ATCC Deposit No. HB-12119, and an anti-CD80 antibody or a CD80-binding fragment thereof which blocks CD80/CD28 signaling without blocking CD80/CTLA4 signaling.

12-15. (canceled)

16. The method according to claim 1, wherein the anti-CD80 antibody is galiximab.

17. The method of claim 1, wherein the immunomodulatory effects are selected from the group consisting of inhibiting activation of regulatory T cells, inhibiting activation of Th2 helper cells, inhibiting activation of both regulatory T cells and Th2 cells, inhibiting proliferation of regulatory T cells, inhibiting proliferation of Th2 helper cells, inhibiting proliferation of both regulatory T cells and Th2 cells, blocking CD80/CD28 signaling without blocking CD80/CTLA4 signaling, reducing production of one or more inflammatory cytokines in the tumor microenvironment, reducing non-malignant CD80-expressing cells in the tumor microenvironment, reducing production of one or more malignant cell survival signals by non-malignant cells of the tumor microenvironment, and enhancing immunity to the malignancy in the subject.

18-20. (canceled)

21. The method of claim 17, wherein the one or more inflammatory cytokines are selected from the group consisting of interleukin-2, interleukin-4, interleukin-8, interleukin-10, interleukin-12, transforming growth factor beta, and interferon-γ.

22. (canceled)

23. The method of claim 17, wherein the CD80-expressing cells are antigen presenting cells, myeloid-derived monocytes, CD14+-expressing monocytes or Tie-2-expressing monocytes.

24. (canceled)

25. The method of claim 17, wherein the one or more survival signals is selected from the group consisting of CD40/CD40L, signaling, interleukin-1, or interleukin-6.

26. (canceled)

27. The method of claim 1, further comprising administering to the subject an anti-cancer agent, wherein the CD80-targeted therapeutic and the anti-cancer agent are administered concurrently or consecutively in either order.

28. The method of claim 27, wherein the second therapeutic agent is selected from the group consisting of cytotoxins, radioisotopes, chemotherapeutic agents, immunomodulatory or immunoregulatory agents, anti-angiogenic agents, anti-proliferative agents, pro-apoptotic agents, cytostatic and cytolytic enzymes, enzyme inhibitors, and tumor vaccines.

29-94. (canceled)

95. The method of claim 1, wherein the CD80-expressing cells are macrophages, dendritic cells, or myeloid-derived suppressor cells.

96-111. (canceled)
